(19) United States
(12) Patent Application Publication
(10) Pub. No.: US 2010/0284949 A1
(43) Pub. Date: Nov. 11, 2010

(54) Water-Containing Composition Which Can Be Used As External Composition

(75) Inventors: Takashi Teshigawara, Kanagawa (JP); Reiji Miyahara, Kanagawa (JP); Takashi Oka, Kanagawa (JP)

Correspondence Address: TOWNSEND & BANTA;c/o PORTFOLIO IP, PO BOX 52050, MINNEAPOLIS, MN, 55402, US

(73) Assignee: SHISEIDO COMPANY, LTD., Tokyo (JP)

(21) Appl. No.: 12/810,183

(22) PCT Filed: Dec. 25, 2008
(86) PCT No.: PCT/JP2008/073603

§ 371 Date: Jun. 23, 2010

(30) Foreign Application Priority Data
Dec. 27, 2007 (JP) 2007-336112

(51) Int. Cl. A61K 8/33; A61Q 17/04

(52) U.S. Cl. 424/59

(57) ABSTRACT

An object of the present invention is to provide means for enhancing ability to solubilize a low water-soluble substance or to disperse the substance in water. The present invention provides a water-containing composition containing water; at least one polyoxyethylene-polyoxypropylene-added phytosterol or phytostanol represented by the following formula (1):

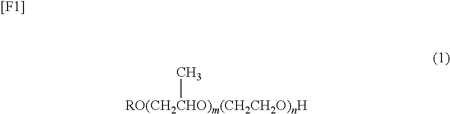

[wherein R represents a phytosterol residue or a phytostanol residue; m is a number from 5 to 100; and n is a number from 5 to 100]; and at least one low water-soluble substance which is in a dissolved state.

### TECHNICAL FIELD

The present invention relates to a water-containing composition that can be employed as a composition for external use (hereinafter may be referred to as an “external-use composition”) in the form of cosmetic composition or external-use agent.

### BACKGROUND ART

Among many substances that are difficult to dissolve in water (hereinafter such a substance may be referred to as a “low water-soluble substance”), quite a lot of substances are useful in the field of external-use compositions for the skin including external-use compositions for the scalp or hair (e.g., cosmetic compositions or external-use agents). Various techniques have been provided for reliably incorporating such a low water-soluble substance into an external-use composition. Typical examples of such a technique include emulsification and solubilization. Dispersion in water or solubilization may be employed as a technique which realizes spreading of a low water-soluble substance on the skin with favorable refreshing sensation. In particular, solubilization is a technique essential for using a low water-soluble substance in the form of, for example, lotion. In general, a low water-soluble substance is solubilized by use of a surfactant such as polyoxyethylene alkyl ether or polyoxyethylene hydrogenated castor oil. However, when a low water-soluble substance having a high molecular weight (in particular, a low water-soluble substance having a bulky structure) is used, much difficulty is encountered in solubilizing a sufficient amount of the low water-soluble substance by use of such a surfactant.

Patent Document 1: Japanese Patent Application Laid-Open (kokai) No. 2005-511586

### DISCLOSURE OF THE INVENTION

### Problems to be Solved by the Invention

Patent Document 1 discloses that ethoxylated phytosterol or phytostanol is used as means for dissolving a low water-soluble pharmaceutical drug. Ethoxylated phytosterol is used as a solubilizing agent, but, due to its poor ability to solubilize the drug, the agent poses a problem in that it requires a large amount of a surfactant for solubilizing the drug.

In view of the foregoing, an object of the present invention is to provide means for enhancing ability to solubilize a low water-soluble substance or to disperse the substance in water.

### Means for Solving the Problems

In order to achieve the aforementioned object, the present inventors have conducted studies on an ingredient which exhibits excellent ability to solubilize a low water-soluble substance or to disperse the substance in water, and have found that polyoxyethylene-polyoxypropylene-added phytosterol or phytostanol exhibits very excellent ability to solubilize a low water-soluble substance or to disperse the substance in water. The present invention has been accomplished on the basis of this finding.

Accordingly, the present invention provides a water-containing composition comprising water; at least one polyoxyethylene-polyoxypropylene-added phytosterol or phytostanol represented by the following formula (1):

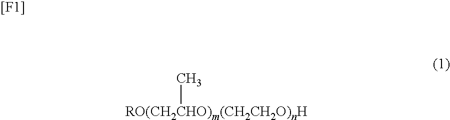

[wherein R represents a phytosterol residue or a phytostanol residue; m is a number from 5 to 100; and n is a number from 5 to 100] (hereinafter such a compound may be referred to as “compound (1)”); and at least one low water-soluble substance which is in a dissolved state (hereinafter the water-containing composition may be referred to as “the water-containing composition of the present invention”). As used herein, the term “water-containing composition” refers to a composition containing water. As used herein, the term “dissolved state” refers to the case where, when a low water-soluble substance contained in a composition is visually observed under no influence of other ingredients, the substance is found to be uniformly present therein in a transparent or semi-transparent state. The “transparent or semi-transparent state” may be, for example, a state where a low water-soluble substance is thermodynamically stable (i.e., “solubilization state”), or a state where a low water-soluble substance is dispersed in the form of fine particles in the aqueous phase (i.e., “water dispersion state”).

Typically, the water-containing composition of the present invention may be employed as an external-use composition that can be applied to the skin, scalp, or hair, or may be employed as a base of the composition. Specific product forms of such an external-use composition include cosmetic compositions and external-use agents. Both “cosmetic composition” and “external-use agent” are in a form that can be applied to the skin, scalp, or hair, and the concepts of these words mutually overlap (in particular, the term “cosmetic composition” encompasses an external-use agent). The case where the external-use composition is employed as an external-use agent rather than a cosmetic composition corresponds to a case where the external-use agent is not a “cosmetic product” for the primary purpose of beauty, but a “quasi-drug” or “drug” for the primary purpose of healthcare.

### EFFECTS OF THE INVENTION

According to the present invention, there is provided very excellent means for solubilizing a low water-soluble substance.

### BEST MODES FOR CARRYING OUT THE INVENTION

As described above, the water-containing composition of the present invention contains compound (1) together with water and a low water-soluble substance. As described hereinbelow, compound (1) contained in the water-containing composition of the present invention may be only one compound represented by the following formula (1), or a mixture of two or more different compounds represented by the following formula (1):

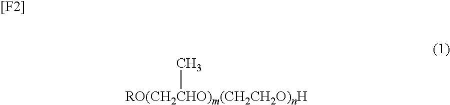

[wherein R represents a phytosterol residue or a phytostanol residue; m is a number from 5 to 100; and n is a number from 5 to 100].

No particular limitation is imposed on the phytosterol, which is a compound that provides a phytosterol residue represented by R in compound (1), and is a plant-derived sterol (F. D. Gunstone and B. G. Herslof, A Lipid Glossary, The Oily Press, Air, 1992). Examples of the phytosterol residue represented by R include a sitosterol residue, a campesterol residue, a stigmasterol residue, a brassicasterol residue, an avenasterol residue, and an ergosterol residue. As described above, compound (1) contained in the water-containing composition of the present invention may be a mixture of two or more compounds having different phytosterol residues represented by R.

Similarly, no particular limitation is imposed on the phytostanol, which is a compound that provides a phytostanol residue represented by R in compound (1), and is obtained through hydrogenation (or saturation) of the corresponding phytosterol. Examples of the phytostanol residue represented by R include sitostanol, campestanol, stigmastanol, brassicastanol, avenastanol, and ergostanol. As described above, compound (1) contained in the water-containing composition of the present invention may be a mixture of two or more compounds having different phytostanol residues represented by R.

Compound (1) contained in the water-containing composition of the present invention may be a mixture of at least one compound having a phytosterol residue represented by R and at least one compound having a phytostanol residue represented by R.

In compound (1), m (i.e., the number representing the polyoxypropylene chain length) is 5 to 100, preferably 5 to 50, and n (i.e., the number representing the polyoxyethylene chain length) is 5 to 100, preferably 10 to 50.

The ratio of m to n is preferably 1:50 to 50:1, particularly preferably 1:5 to 5:1, from the viewpoints of suppressing stickiness of the water-containing composition of the present invention (i.e., providing a favorable sensation upon use thereof) and securing better stability over time.

Compound (1), which has the aforementioned chemical structure, may be produced through a customary method. For example, compound (1) may be readily produced through the following procedure: propylene oxide is added to phytosterol in the presence of a catalyst (i.e., first-step addition reaction); unreacted matter is removed; ethylene oxide is added to the resultant product (i.e., second-step addition reaction); and unreacted matter is again removed, followed by neutralization, dehydration, deodorization, and filtration.

The amount of compound (1) contained in the water-containing composition of the present invention is 10 mass % or less, preferably 3 mass % or less, on the basis of the entirety of the composition. The amount of compound (1) greatly depends on the relationship between the amount thereof and that of the low water-soluble substance contained in the composition. The ratio by mass of compound (1) to low water-soluble substance is preferably 1 or more, particularly preferably 3 or more (the upper limit of this ratio is determined on the basis of the upper limit of the amount of compound (1)). A suitable lower limit of the amount of compound (1) may be determined on the basis of this ratio by mass.

The water-containing composition of the present invention must contain at least one low water-soluble substance, which is to be solubilized.

As used herein, the term “low water-soluble substance” literally refers to a substance which is insoluble or difficult to dissolve in water. No particular limitation is imposed on the solubility of the low water-soluble substance in a solvent other than water.

The low water-soluble substance selected may be, for example, a low water-soluble UV-absorbing agent or medicinal ingredient.

Examples of the low water-soluble UV-absorbing agent include triazine UV-absorbing agents (e.g., bisresorcinyltriazine, bisethylhexyloxyphenol methoxyphenyltriazine (2,4-bis{[4-(2-ethylhexyloxy)-2-hydroxyl]phenyl}-6-(4-methoxyphenyl) 1,3,5-triazine), and octyl triazone (2,4,6-tris[4-(2-ethylhexyloxycarbonyl)anilino]1,3,5-triazine);

benzoic acid UV-absorbing agents (e.g., p-aminobenzoic acid (hereinafter abbreviated as “PABA”), PABA monoglycerin ester, N,N-dipropoxy-PABA ethyl ester, N,N-diethoxy-PABA ethyl ester, N,N-dimethyl-PABA ethyl ester, N,N-dimethyl-PABA butyl ester, and N,N-dimethyl-PABA ethyl ester);

anthranilic acid UV-absorbing agents (e.g., homomethyl-N-acetyl anthranilate);

salicylic acid UV-absorbing agents (e.g., amyl salicylate, menthyl salicylate, homomethyl salicylate, octyl salicylate, phenyl salicylate, benzyl salicylate, and p-isopropanolphenyl salicylate);

cinnamic acid UV-absorbing agents (e.g., octyl cinnamate, ethyl-4-isopropyl cinnamate, ethyl-2,4-diisopropyl cinnamate, methyl-2,4-diisopropyl cinnamate, propyl p-methoxycinnamate, isopropyl p-methoxycinnamate, cyclohexyl p-methoxycinnamate, ethyl-α-cyano-β-phenyl cinnamate, and 2-ethylhexyl-α-cyano-β-phenyl cinnamate);

benzophenone UV-absorbing agents (e.g., 2-[4-(diethylamino)-2-hydroxybenzoyl]benzoic acid hexyl ester, 2,4-dihydroxybenzophenone, 2,2′-dihydroxy-4-methoxybenzophenone, 2,2′-dihydroxy-4,4′-dimethoxybenzophenone, 2,2′,4,4′-tetrahydroxybenzophenone, 2-hydroxy-4-methoxybenzophenone, 2-hydroxy-4-methoxy-4′-methylbenzophenone, 2-hydroxy-4-methoxybenzophenone-5-sulfonic acid salt, 4-phenylbenzophenone, 2-ethylhexyl-4′-phenyl-benzophenone-2-carboxylate, 2-hydroxy-4-n-octoxybenzophenone, and 4-hydroxy-3-carboxybenzophenone);

phenyl acrylate UV-absorbing agents (e.g., 2-ethylhexyl-2-cyano-3,3-diphenyl acrylate and 2-ethyl-2-cyano-3,3-diphenyl acrylate);

phenylbenzimidazole derivatives (e.g., phenylbenzimidazole sulfonic acid and disodium phenyl dibenzimidazole tetrasulfonate);

dibenzoylmethane UV-absorbing agents (e.g., 4-tert-butyl-4′-methoxydibenzoylmethane);

camphor derivatives (e.g., 4-methylbenzylidene camphor, terephthalylidene dicamphor sulfonic acid, 3-(4′-methylbenzylidene)-d,l-camphor, and 3-benzylidene-d,l-camphor);

phenylbenzotriazole derivatives (e.g., hydroxy-(ethylhexyl)phenoxybenzotriazole, methylenebis-benzotriazolyltetramethylbutylphenol, 2-(2′-hydroxy-5′-methylphenyl)benzotriazole, 2-(2′-hydroxy-5′-t-octylphenyl)benzotriazole, 2-[2-hydroxy-4-(2-ethylhexyl)phenoxy]-2H-benzotriazole);

benzalmalonate derivatives (e.g., dimethicone benzalmalonate);

2-phenyl-5-methylbenzoxazole; dibenzaladine; dianisoylmethane; 4-methoxy-4′-t-butyldibenzoylmethane; and 5-(3,3-dimethyl-2-norbornylidene)-3-pentan-2-one.

Examples of the low water-soluble medicinal ingredient include anti-inflammatory agents (e.g., mefenamic acid, flufenamic acid, indomethacin, and ibuprofen); drugs for androgenetic alopecia (e.g., finasteride and minoxidil); antihypertensives (e.g., felodipine, nicardipine hydrochloride, amlodipine besylate, nifedipine, verapamil, candesartan cilexetil, and tocopherol nicotinate); antilipemic drugs (e.g., clofibrate, tocopherol nicotinate, probucol, and cholestyramine); antidiabetics (e.g., glibenclamide and tolbutamide); antifungal drugs (e.g., griseofulvin and miconazole); therapeutic drugs for cardiac diseases (e.g., atenolol, propranolol, and lidocaine); antineoplastic drugs (e.g., bicalutamide and medroxyprogesterone acetate); anesthetics (e.g., lidocaine and propofol); corticosteroids (e.g., prednisolone and dexamethasone); antiphlogistics (e.g., salicylic acid derivatives and hinokitiol); activators (e.g., royal jelly and cholesterol derivatives); and blood circulation promoting agents (e.g., nonylic acid vanillylamide, benzyl nicotinate, β-butoxyethyl nicotinate, capsaicin, zingerone, ichthammol, α-borneol, and tocopherol nicotinate).

Other examples of the low water-soluble substance include low water-soluble vitamins, lipids, proteins, peptides, fatty acids, and low water-soluble antioxidants.

Among the aforementioned low water-soluble substances, a low water-soluble substance having, in the chemical structure thereof, two or more 6-membered rings is preferably employed. Examples of such a low water-soluble substance include UV-absorbing agents such as bisethylhexyloxyphenol methoxyphenyltriazine, octyl triazone, and 2-[2-hydroxy-4-(2-ethylhexyl)phenoxy]-2H-benzotriazole. Of these, bisethylhexyloxyphenol methoxyphenyltriazine exhibits an excellent solubilization promoting effect when used in combination with compound (1).

Examples of the low water-soluble substance having two or more 6-membered rings include anti-inflammatory drugs such as mefenamic acid, flufenamic acid, and indomethacin; drugs for androgenetic alopecia, such as finasteride and minoxidil; and antihypertensives such as felodipine, nicardipine hydrochloride, and tocopherol nicotinate.

No particular limitation is imposed on the amount of the low water-soluble substance contained in the water-containing composition of the present invention, so long as the low water-soluble substance can be maintained in a solubilized state in the composition at ambient temperature. The relationship between the amount of the low water-soluble substance and that of compound (1) is as described above. The specific amount of the low water-soluble substance may be determined in consideration, in addition to the above-described relationship, of the appropriate amount of the substance according to the nature thereof.

The amount of water contained in the water-containing composition of the present invention is the balance of the entire composition containing compound (1) and the low water-soluble substance, or the balance of the entire composition containing compound (1), the low water-soluble substance, and a common ingredient incorporated into the composition.

An embodiment of the water-containing composition of the present invention contains the aforementioned essential ingredients; i.e., water, a low water-soluble substance, and compound (1). By virtue of the presence of compound (1), the low water-soluble substance, which is originally difficult to dissolve in water, is readily solubilized. Preferably, the water-containing composition of the present invention is produced by, for example, dissolving a low water-soluble substance in compound (1) to thereby prepare a portion, and mixing the portion with water for solubilization of the low water-soluble substance.

The water-containing composition of the present invention containing only the aforementioned essential ingredients may be provided as an external-use composition used in the form of cosmetic composition or external-use agent. The external-use composition may be applied to the skin (including the scalp and hair) for providing one or more effects of the solubilized low water-soluble substance (e.g., medicinal effect and UV absorbing effect).

The water-containing composition of the present invention containing only the aforementioned essential ingredients may be employed as a base for preparing an external-use composition into which a common ingredient other than the essential ingredients is incorporated. Specifically, a final external-use composition of interest may be produced by firstly producing the water-containing composition of the present invention, and secondly incorporating a common ingredient into the composition.

Such a common ingredient is selected in consideration of, for example, provision of medicinal effects, coloration, regulation of production parameters, adjustment of ionic strength, adjustment of pH, or stability of a drug product. Examples of the common ingredient include an excipient, a buffer, a salt, an antioxidant, a preservative, a perfume, a dye, a surfactant, a water-soluble vitamin, and a water-soluble drug. The common ingredient employed is generally a water-soluble substance. However, the common ingredient employed may be a low water-soluble substance, depending on the form of a final product. For example, the common ingredient may be a liquid oil ingredient having no 6-membered ring in the structure.

A final external-use composition of interest may be produced without using the above-described production process for an embodiment of the water-containing composition of the present invention containing only the essential ingredients. In such a case, the final external-use composition of interest may be produced through, for example, the following process: a water-soluble ingredient selected as a common ingredient is dissolved in water to thereby prepare an aqueous phase; and the aqueous phase is mixed with a portion prepared by dissolving a low water-soluble substance in compound (1), to thereby solubilize the low water-soluble substance. However, the method for producing the final external-use composition of interest is not limited to this production process, and may be appropriately selected or devised in consideration of the nature of an essential ingredient or selected common ingredient. As described above, the water-containing composition of the present invention may be provided in the form of water dispersion in which fine particles of a low water-soluble substance are dispersed in an aqueous phase.

### Examples

The present invention will next be described in more detail by way of examples, which should not be construed as limiting the invention thereto. Unless otherwise specified, the amount of an ingredient is represented by “mass %” with respect to the total amount of a composition into which the ingredient is incorporated.

### Test Examples

A low water-soluble substance was mixed with and dissolved in compound (1) or another surfactant through a customary method. The resultant portion was mixed with water, to thereby prepare a test sample shown in Tables 1 to 6 (Examples or Comparative Examples). The sample was evaluated according to the below-described criteria. The results of samples of each test system are shown in the corresponding table. In each table, blanks in rows corresponding to the amounts of ingredients incorporated represent “0 mass %.”

(1) Test Immediately after Preparation of Test Sample

### (a) Evaluation of Dissolved State

The dissolved state of each test sample was visually evaluated according to the following criteria:

AA: transparent;

BB: generally transparent;

CC: turbid to semi-transparent; and

DD: phase separation observed.

### (b) Evaluation of Transparency

The transparency of each test sample was evaluated through measurement of L value. L value was determined by means of a spectrophotometer (UV-160, product of Shimadzu Corporation). The transparency (L value) of each test sample was evaluated on the basis of the transparency of distilled water as a control (L value=100) according to the following criteria:

90 or more: transparent;

70 to 90: generally transparent;

less than 70: turbid to semi-transparent; and

not determined: phase separation observed.

### (c) Sensation Upon Use

The sensation upon use of each test sample was evaluated by 10 expert panelists according to the following criteria:

O: 5 or more of the 10 expert panelists assessed that the test sample exhibited no stickiness;

Δ: 3 to 4 of the 10 expert panelists assessed that the test sample exhibited no stickiness; and

x: 0 to 2 of the 10 expert panelists assessed that the test sample exhibited no stickiness.

### (2) Test on Change Over Time

Each of the above-prepared test samples was stored at 50° C. for one month, and the L value of the thus-stored sample was measured in a manner similar to that described above. The stability over time of each test sample was evaluated on the basis of the difference between the L value determined immediately after preparation and the L value determined after one-month storage. The following criteria were used for evaluation. The L values determined after one-month storage are shown in the corresponding tables.

AA: difference in L value ≦2 (±);

BB: difference in L value ≦5 (±);

CC: difference in L value ≦10 (±); and

DD: difference in L value ≧10 (±), or phase separation observed.

### <Test System 1>

TABLE 1

| | Comp. | Comp. | Comp. | | | | |
|---|---|---|---|---|---|---|---|
| | Ex. 1 | Ex. 2 | Ex. 3 | Ex. 1 | Ex. 2 | Ex. 3 | Ex. 4 |
| Ion-exchange water | to 100 | to 100 | to 100 | to 100 | to 100 | to 100 | to 100 |
| POE hydrogenated | 0.3 | — | — | — | — | — | — |
| castor oil | | | | | | | |
| POE-POP-added decyl | — | 0.3 | — | — | — | — | — |
| tetradecyl ether | | | | | | | |
| POE (30) phytosterol | — | — | 0.3 | — | — | — | — |
| POE (25)-POP (20) phytosterol | — | — | — | 0.3 | 1.5 | 0.4 | 1.0 |
| Bisethylhexyloxyphenol | 0.1 | 0.1 | 0.1 | 0.1 | 0.1 | — | — |
| methoxyphenyltriazine | | | | | | | |
| Finasteride | — | — | — | — | — | 0.1 | 0.1 |
| Dissolved state | DD | DD | CC | AA | AA | AA | AA |
| (immediately | | | | | | | |
| after preparation) | | | | | | | |
| L value (immediately | 6 | 4 | 42 | 96 | 99 | 97 | 98 |
| after preparation) | | | | | | | |

As shown in Table 1, the surfactant contained in the test sample of Comparative Example 1 or 2, which has no phytosteryl skeleton, exhibited poor ability to solubilize a low water-soluble substance; i.e., the surfactant was found to be unsuitable for solubilization of the substance.

Although the surfactant contained in the test sample of Comparative Example 3 has a phytosteryl skeleton, the surfactant does not have a polyoxypropylene skeleton. Therefore, the surfactant exhibited poor ability to solubilize a low water-soluble substance; i.e., the surfactant failed to solubilize the substance at a practical level.

Compound (1) contained in each of the test samples of Examples 1 to 4 solubilized a low water-soluble substance at a practically sufficient level.

### <Test System 2>

The test system shown in Table 2 focused on the ratio of the number of EO units (n) to that of PO units (m) in compound (1). Therefore, the ratio is also shown in the table.

TABLE 2

| | Ex. 5 | Ex. 6 | Ex. 7 | Ex. 8 | Ex. 9 | Ex. 10 |
|---|---|---|---|---|---|---|
| Ion-exchange water | to 100 | to 100 | to 100 | to 100 | to 100 | to 100 |
| POE (3)-POP (45) phytosterol | 0.5 | — | — | — | — | — |
| POE (15)-POP (30) phytosterol | — | 0.5 | — | — | — | — |
| POE (40)-POP (20) phytosterol | — | — | 0.5 | — | — | — |
| POE (20)-POP (5) phytosterol | — | — | — | 0.5 | — | — |
| POE (70)-POP (7) phytosterol | — | — | — | — | 0.5 | — |
| POE (100)-POP (2) phytosterol | — | — | — | — | — | 0.5 |
| 2,4,6-tris[4-(2- | 0.1 | 0.1 | 0.1 | 0.1 | 0.1 | 0.1 |
| ethylhexyloxycarbonyl)anilino] | | | | | | |
| 1,3,5-triazine | | | | | | |
| EO/PO ratio of surfactant | 1/15 | 1/3 | 2/1 | 4/1 | 10/1 | 50/1 |
| Dissolved state (immediately after | AA | AA | AA | AA | AA | AA |
| preparation) | | | | | | |
| L value (immediately | 90 | 92 | 97 | 98 | 98 | 99 |
| after preparation) | | | | | | |
| Sensation upon use (immediately after | ◯ | ◯ | ◯ | ◯ | Δ | Δ |
| preparation) | | | | | | |
| Stability over time | BB | AA | AA | AA | AA | AA |
| L value after 1-M storage at 50° C. | 86 | 92 | 97 | 98 | 98 | 99 |

As shown in Table 2, the test sample of Example 5—in which the number (mol) of added polyoxyethylene units is 5 or less, and the ratio of the number (mol) of added polyoxyethylene units to that of added polyoxypropylene units is 1/50 or less—exhibited slightly poor stability over time.

The test sample of Example 10—in which the number (mol) of added polyoxypropylene units is 5 or less, and the ratio of the number (mol) of added polyoxyethylene units to that of added polyoxypropylene units is 50 or more—exhibited stickiness upon use.

The test example of Example 9, in which the ratio of the number (mol) of added polyoxyethylene units to that of added polyoxypropylene units is 5/1 or more, exhibited stickiness upon use.

The test samples of Examples 6 to 8—in which the number (mol) of added polyoxyethylene units is 5 to 100, the number (mol) of added polyoxypropylene units is 5 to 100, and the ratio of the number (mol) of added polyoxyethylene units to that of added polyoxypropylene units is 5/1 to 1/5—exhibited good appearance, sensation upon use, and stability over time.

### <Test System 3>

The test system shown in Table 3 focused on the ratio by mass of compound (1) to low water-soluble substance. Therefore, the ratio is also shown in the table.

TABLE 3

| | Ex. 11 | Ex. 12 | Ex. 13 | Ex. 14 |
|---|---|---|---|---|
| Ion-exchange water | to 100 | to 100 | to 100 | to 100 |
| POE (40)-POP (30) phytosterol | 3.0 | 1.5 | 0.8 | 0.4 |
| 4-tert-Butyl-4′- | 0.2 | 0.3 | 0.4 | 0.8 |
| methoxydibenzoylmethane | | | | |
| Ratio of compound (1)/low | 15/1 | 5/1 | 2/1 | 1/2 |
| water-soluble substance | | | | |
| Evaluation of appearance | AA | AA | AA | BB |
| L value | 98 | 99 | 96 | 85 |
| Sensation upon use | ◯ | ◯ | ◯ | ◯ |
| Stability over time | AA | AA | AA | BB |
| L value after 1-M | 98 | 99 | 95 | 81 |
| storage at 50° C. | | | | |

The test sample of Example 14, in which the ratio by mass of compound (1) to low water-soluble substance is 1 or less, exhibited a slightly semi-transparent state.

The test samples of Examples 11 to 13, in which the ratio by mass of compound (1) to low water-soluble substance is 1 or more, exhibited good appearance, sensation upon use, and stability.

### <Test System 4>

TABLE 4

| | Ex. 15 | Ex. 16 | Ex. 17 | Ex. 18 | Ex. 19 |
|---|---|---|---|---|---|
| Ion-exchange water | to 100 | to 100 | to 100 | to 100 | to 100 |
| POE (30)-POP (30) phytosterol | 0.4 | 0.4 | 0.4 | 0.4 | 0.4 |
| Vitamin E acetate | 0.2 | — | — | — | — |
| 2-Ethylhexyl p-methoxycinnamate | — | 0.2 | — | — | — |
| Hexyl diethylaminohydroxybenzoylbenzoate | — | — | 0.2 | — | — |
| 2-[2-Hydroxy-4-(2-ethylhexyl)phenoxy]-2H- | — | — | — | 0.2 | — |
| benzotriazole | | | | | |
| 2-Ethylhexyl-2-cyano-3,3-diphenylacrylate | — | — | — | — | 0.2 |
| Evaluation of appearance | AA | AA | AA | AA | AA |
| L value | 95 | 97 | 94 | 98 | 96 |
| Stability over time | BB | BB | AA | AA | AA |
| L value after 1-M storage at 50° C. | 91 | 93 | 94 | 97 | 96 |

Each of the test samples of Examples 15 and 16, which contains a low water-soluble substance having only one 6-membered ring in the chemical structure thereof, exhibited good appearance (i.e., the substance was solubilized by compound (1)), but failed to achieve best results in terms of stability over time.

Each of the test samples of Examples 17 to 19, which contains compound (1) and a low water-soluble substance having two or more 6-membered rings in the chemical structure thereof, exhibited good appearance and stability.

### <Test System 5>

The test system shown in Table 5 employed a formulation (external-use agent) containing common ingredients in addition to essential ingredients of the water-containing composition of the present invention. For preparation of each test sample, the corresponding common ingredients (water-soluble ingredients) were mixed with ion-exchange water to thereby prepare an aqueous phase portion, and subsequently the aqueous phase portion was mixed with a portion prepared by dissolving a low water-soluble substance in compound (1).

TABLE 5

| | Ex. 20 | Ex. 21 | Ex. 22 | Ex. 23 |
|---|---|---|---|---|
| Ion-exchange water | to 100 | to 100 | to 100 | to 100 |
| Ethanol | 5.0 | 5.0 | 5.0 | 5.0 |
| Dynamite glycerin | 3.0 | 3.0 | 3.0 | 3.0 |
| Dipropylene glycol | 2.0 | 2.0 | 2.0 | 2.0 |
| POE (14)-POP (7) | 1.0 | 1.0 | 1.0 | 1.0 |
| dimethyl ether | | | | |
| POE (40)-POP (30) | 0.4 | 0.4 | 0.4 | 0.4 |
| phytosterol | | | | |
| Citric acid | 0.05 | 0.05 | 0.05 | 0.05 |
| Sodium citrate | 0.05 | 0.05 | 0.05 | 0.05 |
| Sodium ascorbate | 2.0 | — | — | — |
| Tranexamic acid | 1.0 | 2.0 | 0.5 | — |
| Potassium | — | — | 1.0 | 1.0 |
| 4-methoxysalicylate | | | | |
| Phenoxyethanol | 0.5 | 0.5 | 0.5 | 0.5 |
| Perfume | 0.01 | 0.01 | 0.01 | 0.01 |
| Red No. 227 | 0.0001 | 0.0001 | 0.0001 | 0.0001 |
| Bisethylhexyloxyphenol | 0.1 | 0.1 | 0.1 | 0.1 |
| methoxyphenyltriazine | | | | |
| Evaluation of appearance | AA | AA | AA | AA |
| L value | 97 | 98 | 96 | 99 |
| Sensation upon use | ◯ | ◯ | ◯ | ◯ |
| Stability over time | AA | AA | AA | AA |
| L value after 1-M | 96 | 98 | 96 | 98 |
| storage at 50° C. | | | | |

As is clear from data on the test samples of Examples 20 to 23, the original effects of the water-containing composition of the present invention are not impeded even in a system containing common ingredients.

### <Test System 6>

The test system shown in Table 6 employed a formulation (hair cosmetic composition) containing common ingredients in addition to essential ingredients of the water-containing composition of the present invention. For preparation of each test sample, the corresponding common ingredients (water-soluble ingredients) were mixed with ion-exchange water to thereby prepare an aqueous phase portion, and subsequently the aqueous phase portion was mixed with a portion prepared by dissolving a low water-soluble substance in compound (1).

TABLE 6

| | Ex. 24 | Ex. 25 | Ex. 26 |
|---|---|---|---|
| Ion-exchange water | to 100 | to 100 | to 100 |
| Ethanol | 10 | 10 | 10 |
| Propylene glycol | 2.0 | 2.0 | 2.0 |
| Dipropylene glycol | 1.0 | 1.0 | 1.0 |
| POE (14)-POP (7) | 1.0 | 1.0 | 1.0 |
| dimethyl ether | | | |
| POE (20)-POP (10) | 0.8 | 0.8 | 0.8 |
| phytosterol | | | |
| Citric acid | 0.05 | 0.05 | 0.05 |
| Sodium citrate | 0.05 | 0.05 | 0.05 |
| Finasteride | 0.1 | 0.1 | — |
| Stearyltrimethylammonium | 0.1 | 0.1 | 0.1 |
| chloride | | | |
| Phenoxyethanol | 0.5 | 0.5 | 0.5 |
| Perfume | 0.01 | 0.01 | 0.01 |
| Bisethylhexyloxyphenol | — | 0.1 | 0.1 |
| methoxyphenyltriazine | | | |
| Evaluation of appearance | AA | AA | AA |
| L value | 99 | 99 | 97 |
| Sensation upon use | ◯ | ◯ | ◯ |
| Stability over time | AA | AA | AA |
| L value after 1-M | 99 | 98 | 97 |
| storage at 50° C. | | | |

As is clear from data on the test samples of Examples 24 to 26, the original effects of the water-containing composition of the present invention are not impeded even in a system containing common ingredients.

1. A water-containing composition comprising
water;
at least one polyoxyethylene-polyoxypropylene-added phytosterol or phytostanol represented by the following formula (1): 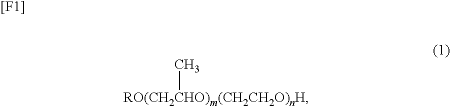
wherein R represents a phytosterol residue or a phytostanol residue, m is a number from 5 to 100, and n is a number from 5 to 100; and
at least one low water-soluble substance which is in a dissolved state.

2. The water-containing composition according to claim 1, which consists of water; the polyoxyethylene-polyoxypropylene-added phytosterol or phytostanol represented by the formula (1); and the low water-soluble substance.

3. The water-containing composition according to claim 1, wherein the ratio of m to n is 1:50 to 50:1.

4. The water-containing composition according to claim 1, wherein the ratio of m to n is 1:5 to 5:1.

5. The water-containing composition according to claim 1, wherein the ratio by mass of polyoxyethylene-polyoxypropylene-added phytosterol or phytostanol (1) to low water-soluble substance is 1 or more.

6. The water-containing composition according to claim 1, wherein the phytosterol residue is one or more phytosterol residues selected from the group consisting of a sitosterol residue, a campesterol residue, a stigmasterol residue, a brassicasterol residue, an avenasterol residue, and an ergosterol residue.

7. The water-containing composition according to claim 1, wherein the phytostanol residue is one or more phytostanol residues selected from the group consisting of a sitostanol residue, a campestanol residue, a stigmastanol residue, a brassicastanol residue, an avenastanol residue, and an ergostanol residue.

8. The water-containing composition according to claim 1, wherein the low water-soluble substance has, in the chemical structure thereof, two or more 6-membered rings.

9. The water-containing composition according to claim 8, wherein the low water-soluble substance is a triazine UV-absorbing agent.

10. The water-containing composition according to claim 1, wherein the low water-soluble substance is one or more species selected from the group consisting of a vitamin, a lipid, a protein, a peptide, a fatty acid, an antioxidant, and a plant- or animal-derived extract.

11. The water-containing composition according to claim 1, which is an external-use composition.

12. The water-containing composition according to claim 11, wherein the external-use composition is in the form of a cosmetic composition or an external-use agent.

13. The water-containing composition according to claim 1, which is employed as a base for producing an external-use composition.
